(19) United States
(12) Patent Application Publication
(10) Pub. No.: US 2004/0018579 A1
(43) Pub. Date: Jan. 29, 2004

(54) Dye-labelled peptide and method

(76) Inventor: Neil Cook, Dover, MA (US)

Correspondence Address: AMERSHAM BIOSCIENCES, PATENT DEPARTMENT, 800 CENTENNIAL AVENUE, PISCATAWAY, NJ, 08855, US

(21) Appl. No.: 10/398,438

(22) PCT Filed: Oct. 3, 2001
(86) PCT No.: PCT/GB01/04462

(30) Foreign Application Priority Data
Oct. 4, 2000 (GB) 0024351.9

(51) Int. Cl. C12Q 1/37; C07K 14/47; C07K 7/08

(52) U.S. Cl. 435/23; 530/324

(57) ABSTRACT

Disclosed is a peptide chain containing one or more dye molecules covalently bonded thereto, characterised in that at least one dye molecule is interposed in the amino sequence forming the peptide chain such that there is at least one amino acid covalently linked to and on each side of the said at least one dye molecule. Also disclosed is an assay method employing the dye-labelled compounds of the invention.

The present invention relates to dye-labelled peptides. In particular, the invention relates to new labelling methods whereby single or multiple fluorescent reporter groups may be incorporated into synthetic polypeptide molecules. Processes for the preparation of the fluorescent dye-labelled peptide derivatives are described, together with their applications in assays employing fluorescence detection.

Biochemical assays utilising labelled peptides in which the reporter group may be, for example, a detectable label such as a fluorescent moiety, are well known. Thus, peptides and proteins can be labelled with fluorescent labelling reagents to provide detectable labels for numerous in vitro assay procedures. The chemistry of peptide labeling is well documented and a wide range of reagents is available for the chemical modification of peptides. Generally, the choice of labelling reagent and the chemistry of labelling will be determined by the amino acid composition of the molecule to be labelled. Particularly preferred are amine reactive fluorescent labelling reagents and thiol reactive labelling reagents. In the first case, the functional group for labelling is a primary amino group which may be derived from the ε-amino group of lysine, or alternatively the peptide amino-terminus. Particular examples of labelling reagents for ε-amino lysine residues include fluorescein isothiocyanate (FITC), fluorescein N-hydroxysuccinimidyl ester, and the mono- and bis-reactive NHS esters of the cyanine dyes. Although relatively few proteins and peptides have free thiol groups (they generally exist as disulphide groups), thiol labelling procedures have proved very useful for labelling proteins and peptides, using thiol-reactive reagents, for example, iodoacetyl and maleimidyl derivatives of fluorescent molecules. For a review and examples of protein labelling using fluorescent labelling reagents, see “Non-Radioactive Labelling, a Practical Introduction”, Garman, A. J. Academic Press,1997; “Handbook of Fluorescent Probes and Research Chemicals”, Haugland, R. P., Molecular Probes Inc., 1992).

Two problems may arise in labelling experiments with peptides and in their subsequent use as labelled probes. Firstly, the fluorescent group may be bulky and large in relation to the peptide to be labelled, and may be attached to the peptide via side chain functional groups. As a result, the fluorescent label may often have an adverse effect on the biological activity of the peptide being labelled. This may be a particular problem with the use of long wavelength fluorophores having extended chromophores. Secondly, not all of the amino acids that are generally available in proteins for covalent attachment of a fluorescent group will necessarily be present in peptides.

Besson et al (Heterocycles, 34(2), 273-291 1992) describe heterobifunctional fluorescent compounds bearing an amino group and a carboxylic group. The authors state that in these compounds, the amine function can react with the carboxylic ends of peptides and the carboxylic group can form bridges with side chains of polymers or lateral chains of amino acids in carrier proteins.

There is a continuing need for alternative labelling strategies for biomolecules such as peptides and proteins. Specifically, new methods are required for the introduction of a fluorescent label into a peptide or into a conjugate between a peptide and another biomolecule such as a carbohydrate. The present invention therefore provides new fluorescent reagents and methods which are of use in labelling peptides and proteins as well as other biomolecules possessing suitable functional groups for attachment of the fluorescent label.

Accordingly, the present invention provides a compound comprising a peptide chain containing one or more dye molecules covalently bonded thereto, characterised in that at least one dye molecule is interposed in the amino acid sequence forming the peptide chain such that there is at least one amino acid covalently linked to and on each side of the said at least one dye molecule.

In a first embodiment of the invention, the dye-labelled peptide chain is of the formula (I):

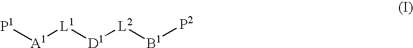

wherein D¹ is a dye molecule;

p¹ is an amino acid or a sequence comprising at least two amino acids and may include one or more functional groups for reaction with other groups;

p² is an amino acid or a sequence comprising at least two amino acids and may include one or more functional groups for reaction with other groups;

A¹ comprises an atom or a group suitable for attaching L¹ to P¹ by means of a covalent linkage;

B¹ comprises an atom or a group suitable for attaching L² to P² by means of a covalent linkage; and

L¹ and L² are each a linker chain and each independently contains from 1-20 linked atoms selected from the group consisting of carbon, nitrogen, oxygen, sulphur and phosphorus and combinations thereof and each L¹ and L² may be independently substituted by one or more groups selected from hydroxyl, halogen, C₁-C₄ alkyl, C₁-C₄ alkoxy, aryl, heteroaryl and aralkyl.

In a second embodiment, the dye-labelled peptide chain includes a dye attached to a terminal amino acid of the peptide sequence. In this embodiment, the dye-labelled peptide chain is suitably of the formula (II):

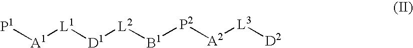

wherein D¹ and D² may be the same or different and are each a dye molecule;

P¹, P², A¹ and B¹ are as hereinbefore defined;

A² comprises an atom or a group suitable for attaching L³ to P² by means of a covalent linkage; and

L¹, L² and L³ are each as hereinbefore defined for L¹ and L².

In a third embodiment, the dye-labelled peptide chain contains more than one dye interposed in the peptide sequence. In this embodiment, the dye-labelled peptide chain is suitably of the formula (III):

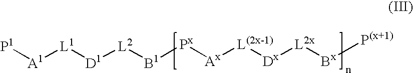

wherein n is an integer and x is (n+1); and

P¹ to P^{(x+1)}, A¹ to A^{x}, B¹ to B^{x}, L¹ to L^{2x} and D¹ to D^{x} are as hereinbefore defined for P¹, P², A¹, B¹, L¹, L², L³, D¹ and D².

In a further embodiment, the dye-labelled peptide chain containing more than one interposed dye may also include a dye attached to a terminal amino acid.

Suitably, n is an integer from 1 to 5. Preferably, n is 1 or 2.

In a preferred embodiment, the dye-labelled peptide chain contains two dyes interposed in the peptide sequence. In this embodiment, the dye-labelled peptide chain is suitably of the formula (IV):

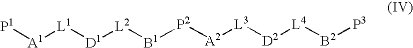

wherein P¹, P², P³, A¹, A², B¹, B², L¹, L², L³, L⁴, D¹ and D² are as hereinbefore defined for P¹, P², A¹, B¹, L¹, L², L³, D¹ and D².

The term peptide chain as used herein is intended to denote oligopeptides, polypeptides, proteins and fragments thereof.

Suitably, P¹ terminates in a functional group for reaction under suitable conditions with A¹. Suitably, P^{x} terminates in a functional group for reaction under suitable conditions with B^{(x-1)}. Alternatively, P^{x} terminates in a first functional group for reaction under suitable conditions with B^{(x-1)} and in a second functional group for reaction under suitable conditions with A^{x}. Suitably, P^{(x+1)} terminates in a functional group for reaction under suitable conditions with B^{x}.

Suitably, the linker chains L¹ to L^{2x} may be selected from linear or branched C₁₋₂₀ alkyl chains, which may optionally contain one or more ether linkages, one or more amide linkages, one or more unsaturated groups, including —CR^{a}═CR^{a}—, —C≡C—, and phenylene which may be substituted with 1, 2, 3 or 4 substituents independently selected from hydroxyl, halogen, C₁-C₄ alkyl, C₁-C₄ alkoxy, aryl, heteroaryl and aralkyl and R^{a} is selected from hydrogen and C₁-C₄ alkyl. Preferably the linker chains L¹ to L^{2x} may be selected from the group consisting of a straight or branched C₁₋₂₀ alkyl chain, a C₂₋₂₀ monoether or polyether and a C₂₋₂₀ atom chain containing up to two secondary amide linkages.

Aryl is an aromatic substituent, for example phenyl or naphthyl, which may be optionally and independently substituted by one or more groups selected from hydroxyl, halogen, C₁-C₄ alkyl and C₁-C₄ alkoxy.

Heteroaryl is a mono- or bicyclic 5-10 membered aromatic ring system containing at least one and no more than 3 heteroatoms which may be selected from N, O and S. The heteroaryl may be optionally and independently substituted by one or more groups selected from hydroxyl, halogen, C₁-C₄ alkyl and C₁-C₄ alkoxy.

Aralkyl is a C₁-C₄ alkyl group substituted by an aryl or heteroaryl group.

Halogen groups are those selected from fluorine, chlorine, bromine and iodine.

In one preferred embodiment, D¹ (and/or D² to D^{x} if present), is a fluorescent dye. In a second preferred embodiment, one or more of D¹ to D^{x} is a fluorescent dye and the remaining D¹ to D^{x} is a non-fluorescent or quenching dye. Preferably, a fluorescent dye and a non-fluorescent dye are at adjacent dye positions, ie. D^{x} is fluorescent and one or both of D^{(x+1)} and D^{(x-1)} is a non-fluorescent or quenching dye, wherein x is hereinbefore defined.

Suitably, D¹ has attached to it linker chains L¹ and L² for linking D¹ respectively to one each of a functional group A¹ and a reactive group B¹. Suitably, each D^{x} (if present), has attached to it linker chains L^{(2x-1)} and L^{2x} for linking D^{x} respectively to at least one functional group A^{x}, and/or reactive group B^{x}, wherein x is hereinbefore defined.

Suitably, groups A¹ to A^{x} are capable of forming covalent linkages with the terminal functional groups respectively of P¹ to P^{x}, and groups B¹ to B^{x} are capable of forming covalent linkages with the terminal functional groups respectively of P² to P^{(x+1)}, wherein x is hereinbefore defined.

Suitably, groups A¹ to A^{x} may be the same or different and may be selected from amino, hydroxyl and sulphydryl, including their protected derivatives. Suitable protecting groups for amino, hydroxyl and sulphydryl groups, such as those for use in peptide synthetic methods, are well known to the skilled person. Preferably, each of groups A¹ to A^{x} is an amino group or a protected amino group such as the N^{α}-t-butyloxycarbonyl (BOC) group or N^{α}-9-fluorenylmethyloxycarbonyl (Fmoc) group. Suitably, groups B¹ to B^{x} may be the same or different and may be selected from a carboxyl (including protected or activated carboxyl groups), isothiocyanate, maleimide, haloacetamide, acid halide, hydrazide, vinylsulphone, dichlorotriazine and phosphoramidite. Preferably, each of groups B¹ to B^{x} is a carboxyl or a protected or activated carboxyl group. Suitable activated carboxyl groups B¹ to B^{x} include succinimidyl ester or sulphosuccinimidyl ester. Suitable protected carboxyl groups are those suitable for peptide synthesis, examples of which will be well known to the skilled person and include ester groups such as t-butyl ester, phenacyl ester and 2,2,2-trichloroethyl ester.

Suitably, the amino acids or amino acid sequences P¹ to P^{(x+1)} flanking D¹ (and D² to D^{x} if present) may be selected from naturally occurring L-amino acids, for example: alanine (Ala or A), arginine (Arg or R), asparagine (Asp or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), m thionine (Met or M), phenylalanine (Phe or F), proline (Pro or P), serine (Ser or S), threonine (Thr or Y), tryptophan (Trp or W), tyrosine (Tyr or Y) and valine (Val or V). However, it is to be understood that the amino acids comprising P¹ to P^{x+1} are not limited to the examples described above and may be represented by analogues of amino acids, including D-amino acids.

In principle, any dye molecule may be used for forming a dye-labelled peptide according to the present invention, providing that the dye contains, or has attached to it, at least one each of a reactive and/or a functional group capable of forming covalent linkages with the amino acid or amino acid sequence P¹ to P^{(x+1)}. Suitable fluorescent dye moieties D¹ to D^{x} may be selected from fluoresceins, rhodamines, coumarins, derivatives of the bis-pyrromethine boron difluoride dyes, such as 3,3′,5,5′-tetramethyl-2,2′-pyrromethene-1,1′-boron difluoride, sold under the trademark BODIPY™ by Molecular Probes Inc. and disclosed in U.S. Pat. Nos. 4,774,339, 5,187,223, 5,248,782 and 5,274,113 (Haugland and Kang), and cyanine dyes. Particularly preferred fluorescent dyes D¹ to D^{x} for use in the present invention are cyanine dyes having the general formula (V):

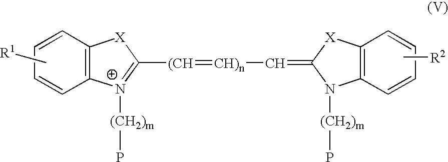

wherein X is selected from C(CH₃)₂, sulphur and oxygen, R¹ and R² are independently selected from the group consisting of CH₂NH₂, SO₃_{⁻}, CH₂COOH and NCS, P is selected from H, SO₃_{⁻}, NH₂ and COOH, n is an integer from 1-3 and m is an integer from 1-5. Cyanine dyes suitable for use in the present invention are disclosed in U.S. Pat. No. 5,268,486 (Waggoner et al) and include the CyDyes™: Cy3, Cy3.5, Cy5, Cy5.5 and Cy7. (CyDye and Cy are trademarks of Amersham Pharmacia Biotech UK Limited.)

Additional cyanine dyes are disclosed in PCT Application No. WO 99/31181 (Waggoner et al) and have the general formula (VI):

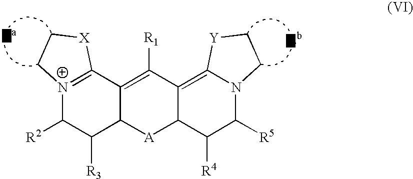

optionally substituted by groups R²—R⁹, wherein groups R⁶, R⁷, R⁸ and R⁹ are attached to the rings containing X and Y or, optionally are attached to atoms of the Z^{a} and Z^{b} ring structures;

R² to R⁹ are the same or different and include —R¹⁰ and -L-R¹⁰ where R¹⁰ is selected from neutral groups that reduce water solubility, polar groups that increase water solubility, functional groups that can be used in labelling reactions, reactive groups, electron donating and withdrawing groups that shift the absorption and emission wavelengths of the fluorescent molecule, lipid and hydrocarbon solubilising groups, and L is selected from the group consisting of a straight or branched C₁₋₂₀ alkyl chain, a C₂₋₂₀ monoether or polyether and a C₂₋₂₀ atom chain containing up to four secondary amide linkages;

A is selected from O, S and NR¹¹ where R¹¹ is the substituted amino radical:

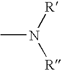

where R′ is selected from hydrogen, a C₁₋₄ alkyl and aryl and R″ is selected from C₁₋₁₈ alkyl, aryl, heteroaryl, an acyl radical having from 2-7 carbon atoms, and a thiocarbamoyl radical;

X and Y may be the same or different and are selected from bis-C₁-C₄ alkyl and C₄-C₅ spiro alkyl substituted carbon, oxygen, sulphur, selenium, CH═CH, and N—W wherein N is nitrogen and W is selected from hydrogen, a group —(CH₂)ₙR¹² where n is an integer from 1 to 26 and R¹² is selected from hydrogen, amino, aldehyde, acetal, ketal, halo, cyano, aryl, heteroaryl, hydroxyl, sulphonate, sulphate, carboxylate, substituted amino, quaternary amino, nitro, primary amide, substituted amide, and groups reactive with amino, hydroxyl, carbonyl, phosphoryl, and sulphydryl groups; and

Z^{a} and Z^{b} each represent a bond or the atoms necessary to complete one, two fused or three fused aromatic rings each ring having five or six atoms, selected from carbon atoms and, optionally, no more than two oxygen, nitrogen and sulphur atoms.

In the embodiment of the invention in which the dye-labelled peptide includes at least two different fluorescent dyes, the labelled peptide may exhibit fluorescence resonance energy transfer (FRET) from a fluorescent donor dye to a fluorescent acceptor dye component. Energy transfer occurs between the electronic excited states of two fluorescent dye molecules when they are in sufficient proximity to each other, wherein the excited-state energy of a donor fluorescent dye is transferred to the acceptor dye. For FRET to occur, the wavelength of the emission maximum of the acceptor dye is typically longer than the wavelength of the emission maximum of the donor dye and a portion of the absorption spectrum of the acceptor dye overlaps a portion of the emission spectrum of the donor, for transferring energy absorbed from the donor dye to the acceptor dye. The result is a decrease in the lifetime and a quenching of fluorescence of the donor species and a concomitant increase in the fluorescence intensity of the acceptor species. Energy transfer efficiency depends on several factors such as spectral overlap, spatial separation between donor and acceptor, relative orientation of donor and acceptor molecules, quantum yield of the donor and excited state lifetime of the donor. In a preferred embodiment of the present invention, the fluorescent donor and acceptor dye molecules may be separated in the peptide chain by a distance that provides efficient energy transfer, preferably better than 75%. Closer proximity of the donor and acceptor fluorophores would enhance energy transfer, since efficiency of energy transfer varies as the inverse 6^{th} power of separation of the centres of the chromophores according to Forster's equation:

ET∝K²Φ_{D}J/R⁶τ_{D}

where ET is the energy transfer rate constant, K² is the relative orientation of donor and acceptor transition moments, Φ_{D} is the quantum yield of the donor molecule, R is the distance between the centres of the donor and acceptor fluorochromes, J is the overlap between the emission spectrum of the donor and the absorption spectrum of the acceptor fluorochromes, and τ_{D} is the excited state lifetime of the donor molecule (Forster, T. “Intermolecular Energy Transfer and Fluorescence”, Ann. Physik., Vol.2, p.55, (1948)).

Alternatively, the dye-labelled peptides of the present invention may employ a fluorescent donor dye and a non-fluorescent (or quenching) acceptor dye in an energy transfer relationship. In such a case, the fluorescence emission of the donor is reduced through quenching by the acceptor. When resonance energy transfer is lost through separation of the fluorescent donor dye and the acceptor dye, the fluorescence emission due to the donor dye is restored.

Suitable non-fluorescent (quenching) acceptor species may be selected from 2,4-dinitrophenyl (DNP), 4-(4-dimethylaminophenyl)azobenzoic acid (DABCYL) and non-fluorescent cyanine dyes as described in PCT WO99/64519 (Birch et al). Particularly preferred non-fluorescent acceptor dyes for use in the invention are cyanine dyes having the structure of formula (VII):

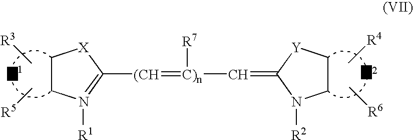

wherein groups R³, R⁴, R⁵ and R⁶ are attached to the rings containing X and Y or, optionally, are attached to atoms of the Z¹ and Z² ring structures and n is an integer from 1-3;

Z¹ and Z² each represent a bond or the atoms necessary to complete one or two fused aromatic rings each ring having five or six atoms, selected from carbon atoms and, optionally, no more than two oxygen, nitrogen and sulphur atoms;

X and Y are the same or different and are selected from bis-C₁-C₄ alkyl- and C₄-C₅ spiro alkyl-substituted carbon, oxygen, sulphur, selenium, —CH═CH— and N—W wherein N is nitrogen and W is selected from hydrogen, a group —(CH₂)ₘR⁸ where m is an integer from 1 to 26 and R⁸ is selected from hydrogen, amino, aldehyde, acetal, ketal, halo, cyano, aryl, heteroaryl, hydroxyl, sulphonate, sulphate, carboxylate, substituted amino, quaternary ammonium, nitro, primary amide, substituted amide, and groups reactive with amino, hydroxyl, carbonyl, carboxyl, phosphoryl, and sulphydryl groups;

at least one of groups R¹, R², R³, R⁴, R⁵, R⁶ and R⁷ is a target bonding group;

any remaining groups R³, R⁴, R⁵, R⁶ and R⁷ groups are independently selected from the group consisting of hydrogen, C₁-C₄ alkyl, OR⁹, COOR⁹, nitro, amino, acylamino, quaternary ammonium, phosphate, sulphonate and sulphate, where R⁹ is selected from H and C₁-C₄ alkyl;

any remaining R¹ and R² are selected from C₁-C₁₀ alkyl which may be unsubstituted or substituted with phenyl the phenyl being optionally substituted by up to two substituents selected from carboxyl, sulphonate and nitro groups;

characterised in that at least one of the groups R¹, R², R³, R⁴, R⁵, R⁶ and R⁷ comprises a substituent which reduces the fluorescence emission of said dye such that it is essentially non-fluorescent.

Suitably, at least one of the groups R³, R⁴, R⁵, R⁶ and R⁷ of the non-fluorescent acceptor dyes according to dyes of formula (VII) is a nitro group which may be attached directly to the rings containing X and Y. Alternatively, a mono- or di-nitro-substituted benzyl group may be attached to the rings containing X and Y, which optionally may be further substituted with one or more nitro groups attached directly to the aromatic rings. Preferably, at least one of groups R¹, R², R³, R⁴, R⁵, R⁶ and R⁷ of the non-fluorescent cyanine dyes of formula (VII) comprises at least one nitro group.

In a preferred embodiment of the invention, the distance R between the centres of D¹ and D² (or D^{x} and D^{(x±1)}), where D¹ and D² (or D^{x} and D^{(x±1)}) are respectively donor and acceptor dyes in an energy transfer relationship and wherein x is hereinbefore defined, may be from 10 to 80 Angstroms. Thus, in a preferred embodiment, the number of amino acid units in P² linking D¹ and D² will suitably be between 2 and 20, preferably between 2 and 10. Preferably, the relative orientation of the transition moments of D^{x} and D^{(x±1)}, wherein x is hereinbefore defined, during the excited state lifetime of the donor and the proximity of the donor and the acceptor dyes are such that there is sufficient energy transfer.

The fluorescent labelled peptides of the present invention may be prepared by chemical coupling of the fluorescent dye derivatives described above to amino acids or to peptide fragments by techniques well known to the skilled person, for example by means of solid phase peptide synthesis methods as described in “Solid Phase Peptide Synthesis”, E. Atherton and R. C. Sheppard, IRL Press 1989. In principle, any amino acid or peptide sequence may be utilised in the formation of fluorescent peptide derivatives of the present invention. The nature and stereochemistry of amino acids utilised in solid phase synthesis techniques is not material to the invention. Rather, the invention discloses the use of fluorescent dye molecules which have been adapted to be suitable for linking to an amino acid or to a peptide chain, and peptides or protein molecules incorporating such dyes. As is known, synthesis of peptides by solid phase techniques is based upon the sequential addition of protected amino acids linked (optionally through a linker group) to a solid phase support. In one commonly employed method, the α-amino group (and side chain amino groups, if any) are suitably protected with acid labile or base labile protecting groups as discussed above. Following addition and coupling of the first amino acid residue, the α-amino protecting group is removed. The chain is extended by the sequential addition of further protected amino acid derivatives or peptide fragments and/or suitably derivatised and protected fluorescent or quencher dye derivatives. In this way, a dye-labelled peptide according to the invention may be constructed by sequential addition of amino acids or a fluorescent dye derivative so as to prepare a peptide containing the desired amino acid sequence, interspersed with one or more fluorescent or quencher dye molecules. Suitably, one (but not both), of the functional group and the reactive group of the dye molecule, as defined hereinbefore, is protected prior to coupling of the dye to the amino-terminus of the preceding amino acid or peptide chain. Once coupled, the protecting group may be removed by methods that are well known to the skilled person. The next protected amino acid or peptide unit is then added using either a coupling reagent or activated amino acid derivative as is known. By this means, a fluorescent dye-labelled peptide derivative of desired amino acid sequence and containing one or more fluorescent dye molecules may be synthesised.

The peptides labelled according to the present invention may be used as fluorescence labels in assays employing fluorescence detection and measurement, for example, by steady state fluorescence intensity, fluorescence lifetime or fluorescence polarisation. Thus, in a further embodiment, there is provided a method for detecting the presence of a biological material which method comprises use of a compound according to formulas (I), (II), (III) or (IV). For example, a fluorescent labelled peptide derivative may be used as probe for a target material, eg. a cell surface receptor, for which the labelled peptide is specific. By this means, the density of cell surface receptors may be determined by the intensity of the fluorescence of the cells when visualised by means of a microscope.

Alternatively, the fluorescence labelled derivative may be used in assay methods involving fluorescence detection. Thus, in a still further embodiment of the present invention, an assay method may comprise separating two components which are in an energy transfer relationship each of said components comprising a peptide or protein or fragment thereof, the first component being labelled with a fluorescent donor dye and the second component being labelled with a non-fluorescent acceptor dye wherein at least one of said dye molecules is interposed in the amino acid chain forming the peptide or protein or fragment such that there is at least one amino acid covalently linked to and on either side of the said at least one dye molecule, and detecting the presence of the first component by measuring emitted fluorescence. Dye-labelled peptides incorporating two such dye moieties may be used in protease assays in which the cleavage of a peptide or protein by a protease is detected by a change in fluorescence intensity. In such assays, the enzyme substrate (peptide, or protein or fragment thereof) may include a sequence whose structure combines the fluorescent donor dye molecule with the non-fluorescent or quenching acceptor dye, covalently bound to the peptide substrate at either side of the substrate bond to be cleaved. The substrate joins the fluorescent donor and the acceptor moieties in close proximity and the intrinsic fluorescence of the donor is reduced through quenching by the acceptor due to resonance energy transfer between the pair of dyes. Resonance energy transfer becomes insignificant when the distance between the donor and acceptor moieties is greater than about 100 Angstroms. Cleavage of the substrate by the protease results in the separation between donor and acceptor dyes and concomitant loss of resonance energy transfer. The fluorescence signal of the donor fluorescent dye increases, thereby enabling accurate measurement of the cleavage reaction. It is to be understood that in the present invention, either D¹ or D² (or D^{x} or D^{(x±1)}), may serve as the donor component, the remaining dye D¹ or D² (or D^{x} or D^{(x±1)}), being the acceptor. The donor and acceptor species and their position of incorporation into the peptide chain are selected such that proteolytic enzyme cleavage of the substrate is not affected to any significant degree. Such assays may be used in high throughput screening applications, including those in which compounds are to be screened for their inhibitory effects, potentiation effects, agonistic, or antagonistic effects on the reaction under investigation.

Briefly, an assay for the detection of proteolytic enzyme activity may be configured as follows. A reaction mixture is prepared by combining a protease enzyme and a peptide substrate according to the present invention which combines a fluorescent donor dye molecule with a quenching dye attached to the substrate at either side of the substrate bond to be cleaved, as described above. A known or a putative protease inhibitor compound may be optionally included in the reaction mixture. Typically the reaction is performed in buffered solution and the reaction is allowed to proceed to completion. The progress of the reaction may be monitored by observing the steady state fluorescence emission due to the fluorescent donor dye, which is recorded using a spectrofluorimeter.

The invention is further illustrated by reference to the following examples and figures.

### FIGURES

FIG. 1 illustrates the course of a trypsin cleavage assay of protease substrate (Compound A), compared with a control dual-labelled fluorescent peptide substrate (Compound B) according to Example 3.1.

FIG. 2 shows the results after subtraction of no-enzyme blanks.

FIG. 3 illustrates an AspN catalysed cleavage of Compound A according to Example 3.2.

### EXAMPLES

1. Preparation of Cyanine Dye Labelled Protease Substrate: Cy5Q -Asp-Glu-Val-Asp-Arg-Ser-Gly-Ser-Gly-Ser-Cy3-Ala-Leu-Thr-OH (Compound A)

1.1 Preparation of 2-{(E)-3-[5-(carboxymethyl)-3,3-dimethyl-1-(4sulfobutyl)1,3-dihydro-2H-indol-2-ylidene]-1-propenyl}-5-({[(9H-fluoren-9-ylmethoxy)carbonyl]amino}methyl)-3,3-dimethyl-1-(4-sulphobutyl)-3H-indolium (Compound 1)

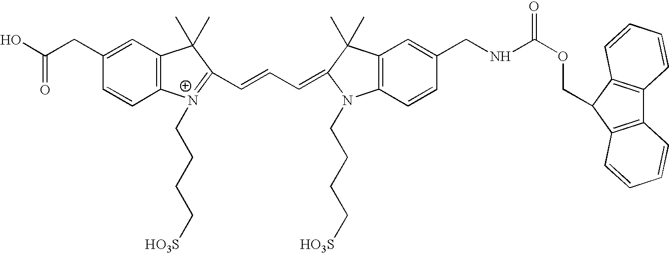

i) 5-[(1,3-Dioxo-1,3-dihydro-2H-isoindol-2-yl)methyl]-2,3,3-trimethyl-3H-indolenine

To a stirred solution of 2,3,3-trimethylindolenine (20 g, 126 mmol) in concentrated sulfuric acid (100 ml) at RT was added in portions, over forty minutes, N-(hydroxymethyl)phthalimide (20 g, 114 mmol). The solution was stirred at RT for 70 hr. The reaction mixture was poured onto ice (800 g) and then made basic by adding concentrated ammonia (˜300 ml) to pH 12. The precipitate which formed was filtered off and washed with water. The product was dried in vacuo to give an off white powder (45.05 g). MS (MALDI-TOF); found 318(M⁺); [theoretical (C₂₀H₁₈N₂O₂) 318].

ii) 5-8 (1,3-Dioxo-1,3-dihydro-2H-isoindol-2-yl)methyl]-2,3,3-trimethyl-1-(4-sulphobutyl)-3H-indolium, Inner Salt

5-[(1,3-Dioxo-1,3-dihydro-2H-isoindol-2-yl)methyl]-2,3,3-trimethyl-3H-indolenine (5 g, 1 5.7 mmol), butyronitrile (80 ml) and 1,4-butane sultone (4.3 ml, 31.6 mmol) were heated together at 120° C. for 72 hr. The reaction was cooled to room temperature and the product filtered off, washed with butyronitrile (2×50 ml) and dried in vacuo. The product was obtained as a beige power (4.05 g). MS (MALDI-TOF) found 455 (MH⁺); theoretical (C₂₄H₂₆N₂O₅S) 454].

iii) 2,3,3-Trimethyl-3H-indol-5-yl-acetic Acid

2,3,3-Trimethyl-3H-indol-5-yl-acetic acid was prepared by the method of Southwick et al, Org. Prep. Proceed. Int., 20, 274-284 (1989).

iv) 5-(Carboxymethyl)-2,3,3-trimethyl-1-(4-sulphobutyl)-3H-indolium, Inner Salt

2,3,3-Trimethyl-3H-indol-5-yl- acetic acid (5 g, 22.1 mmol), butyronitrile (50 ml) and 1,4-butane sultone (4.8 ml, 35.3 mmol) were heated together at 120° C. for 72 hr. The reaction was cooled to room temperature and the product filtered off, washed with ethyl acetate (2×50 ml) and dried in vacuo. The product was obtained as a beige powder (4.91 g). MS (MALDI TOF) found 355 (MH⁺); [theoretical (C₁₇H₂₃NO₅S) 354].

v) 2-[(E)-2-Anilinoethenyl]-3,3-dimethyl-5-(1,3-dioxo-1,3-dihydro-2H-isoindol-2-yl)-1-(4-sulphobutyl)-3H-indolium, Salt

5-[(1,3-Dioxo-1,3-dihydro-2H-isoindol-2-yl)methyl]-2,3,3-trimethyl-1-(4-sulphobutyl)-3H-indolium, inner salt (5 g, 9 mmol), N,N-diphenylformamidine (2.75 g, 14 mmol) and acetic acid (50 ml) were heated together at 140° C. for 18 hr. The reaction was allowed to cool to room temperature. The product was purified by HPLC (Dynamax C18 column (50×4.14 cm); flow rate 25 ml/min; gradient of 0 to 100% B over 90 mins (eluent A=0.1% TFA in water and eluent B=0.1% TFA in acetonitrile); detection at 450 nm), the retention time of the product was 65 mins. The fractions containing the desired product were pooled and the solvent removed under reduced pressure. The product was obtained as an orange solid (1.69 g). MS (MALDI TOF) found 558 (M⁺); [theoretical (C₃₁H₃₁N₃O₅S) 558].

vi) 2-{(E)-3-[5-(1,3-Dioxo-1,3-dihydro-2H-isoindol-2-yl)methyl)-3,3dimethyl-1-(4-sulphobutyl)1,3-dihydro-2H-indol-2-ylidene]-1-propenyl}5-(carboxymethyl)-3,3-dimethyl-1-(4-sulphobutyl)-3H-indolium, Salt

5-(Carboxymethyl)-2,3,3-trimethyl-1-(4-sulphobutyl)-3H-indolium, inner salt (0.5 g, 1.41 mmol), 2-[(E)-2-anilinoethenyl]-3,3-dimethyl-5-(1,3dioxo-1,3-dihydro-2H-isoindol-2-yl)-1-(4-sulphobutyl)-3H-indolium, salt (0.5 g), acetic acid (8 ml) pyridine (8 ml) and acetic anhydride (2 ml) were stirred together at room temperature for 24 hr. The reaction solvents were removed under reduced pressure and the residue purified by HPLC (Dynamax C18 column (50×4.14cm); flow rate 25 ml/min; gradient of 0 to 100% B over 90 mins (eluent A=0.1% TFA in water and eluent B=0.1% TFA in acetonitrile); detection at 550 nm), the retention time of the product was 70 mins. The fractions containing the desired product were pooled and the solvent removed under reduced pressure. The product was obtained as a magenta solid (505 mg). MS (MALDI TOF) found 818 (M⁺); [theoretical (C₄₂H₄₇N₃O₁₀S₂) 818].

vii) 2-{(E)-3-[5-(Aminomethyl)-3,3-dimethyl-1-(4-sulphobutyl)-1,3-dihydro-2H-indol-2-ylidene]-1-propenyl}-5-(carboxymethyl)-3,3-dimethyl-1-(4sulphobutyl)-3H-indolium, Salt

2-{(E)-3-[5-(1,3-Dioxo-1,3-dihydro-2H-isoindol-2-yl)methyl)-3,3dimethyl-1-(4-sulphobutyl)-1′2-dihydro-2H-indol-2-ylidene]-1-propenyl)-5-(carboxymethyl)-3dimethyl-1-(4-sulphobutyl)-3H-indolium, salt (505 mg, 0.9 mmol) and concentrated hydrochloric acid 125 ml) were heated together at 110° C. for 20 hrs. The reaction mixture was cooled to room temperature and the solvent removed under reduced pressure. The residue was purified by HPLC (Dynamax C18 column (50×4.14 cm); flow rate 25 ml/min; gradient of 0 to 100% B over 90 mins (eluent A=0.1% TFA in water and eluent B=0.1% TFA in acetonitrile); detection at 550 nm), the retention time of the product was 62 mins. The fractions containing the desired product were pooled and the solvent removed under reduced pressure. The product was obtained as a magenta solid (285 mg). MS (MALDI TOF) found 688 (M⁺); [theoretical (C₃₄H₄₆N₃O₈S₂) 689].

viii) 2-{(E)-3-[5-(Carboxymethyl)-3,3-dimethyl-1-(4-sulphobutyl) 1,3-dihydro-2H-indol-2-ylidene]1-propenyl}-5-({[(9H-fluoren-9ylmethoxy)carbonyl]amino}methyl)-3,3-dimethyl-1-(4-sulphobutyl)-3H-indolium, Salt (Compound 1)

2-{(E)-3-[5-(Aminomethyl)-3,3-dimethyl-1-(4-sulphobutyl)-1,3-dihydro-2H-indol-2-ylidene]-1-propenyl}-5-(carboxymethyl)-3,3-dimethyl-1-(4sulphobutyl)-3H-indolium, salt (200 mg, 0.29 mmol) was dissolved in dimethylsulphoxide (5 ml). N-(9-fluorenylmethoxycarbonyloxy)-succinimide (250 mg, 0.74 mmol) and diisopropylethylamine (268 μl, 1.7 mmol) were added and the reaction mixture was stirred at room temperature for 16 hrs. Dimethylsulphoxide was removed by washing with diethyl ether to give a magenta residue. The residue was re-dissolved in 10% acetonitrile/water and purified by HPLC (Waters Spherisorb S5ODS2 column (20×250 mm); flow rate 6 ml/min; gradient of 15 to 100% B over 60 mins (eluent A=0.1% TFA in water and eluent B=0.1% TFA in acetonitrile); detection at 550 nm). The fractions containing the desired product were pooled and the solvent removed under reduced pressure. The product was obtained as a magenta solid (136 mg). MS (MALDI TOF) found 909 (M⁺); [theoretical (C₄₉H₅₅N_{3 O}₈S₂) 910].

1.2 Synthesis of Cyanine Dye Labelled Protease Substrate: Cy5Q -Asp-Glu-Val-Asp-Arg-Ser-Gly-Ser-Gly-Ser-Cy3-Ala-Leu-Thr-OH

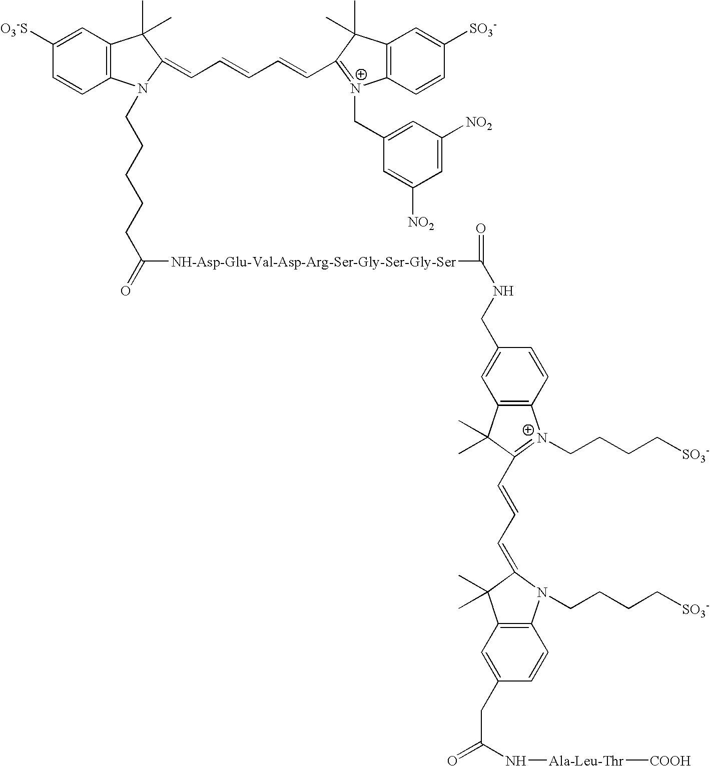

i) Ala-Leu-Thr-Wang Resin

Ala-Leu-Thr-Wang resin was synthesised using a commercially available Perkin-Elmer Model 431A automated peptide synthesiser and FastMoc™ chemistry, following the instrument manufacturer's recommended procedures throughout. The synthesis was performed on a 0.25 millimolar scale. The resin was removed from the peptide synthesiser and dried in vacuo.

ii) Fmoc-Cy3-Ala-Leu-Thr-Wang Resin

Fmoc-protected cyanine dye amino acid analogue (Compound 1) (91 mg, 0.1 mmol) was coupled manually to Ala-Leu-Thr-Wang resin (250 mg, 0.1 mmol) using 7-azabenzotriazol-1-yloxytris(pyrrolidino)phosphonium-hexafluorophosphate (PyAOP) (62.6 mg, 0.12 mmol) and diisopropylethylamine (122 μl, 0.7 mmol) in N-methyl pyrrolidone (NMP) at room temperature for 16 hrs. The reaction solvent was removed and the resin washed with NMP (3×10 ml). The resin was then capped using a standard capping solution (0.5M acetic anhydride, 0.125M diisopropylethylamine, 0.015M hydroxybenzotriazole), washed with N-methylpyrrolidone, dichloromethane and finally diethyl ether before drying in vacuo.

iii) Asp-Glu-Val-Asp-Arg-Ser-Gly-Ser-Gly-Ser-Cy3-Ala-Leu-Thr-Wang Resin

Fmoc-Cy3-Ala-Leu-Thr-Wang resin was returned to the Perkin-Elmer Model 431A automated peptide synthesizer and the final sequence of the peptide built using standard FastMoc™ chemistry, following the instrument manufacturer's recommended procedures throughout. The syntheses were performed on a 0.1 millimolar scale.

iv) Cy5Q-Asp-Glu-Val-Asp-Arg-Ser-Gly-Ser-Gly-Ser-Cy3-Ala-Leu-Thr-OH

2-{5-[1-(5-Carboxypentyl)-3,3-dimethyl-5-sulpho-1,3-dihydro-2H-indol-2-ylidene]-1,3-pentadienyl}-1-(3,5-dinitrobenzyl)-3,3-dimethyl-5-sulfo-3H-indolium, N-hydroxysuccinimidyl ester (Cy5Q NHS ester) was prepared according to the methods described in PCT Application No. WO 99/64519. Cy5Q NHS ester was coupled to Asp-Glu-Val-Asp-Arg-Ser-Gly-Ser-Gly-Ser-Cy3-Ala-Leu-Thr-Wang resin in DMSO containing diisopropylethylamine at room temperature for 12 hrs. The purple resin was filtered off, washed with DMSO, methanol and finally dichloromethane before drying in vacuo. The peptide was cleaved from the solid phase using a mixture of 95% trifluoroacetic acid:2.5% water:2.5% triisopropylsilane. The crude peptide obtained from the cleavage reaction was purified by conventional C18 reverse phase HPLC using a linear gradient of water/acetonitrile (both containing 0.1% trifluoroacetic acid). After purification, the peptide was lyophilised to give a purple fluffy solid. The molecular weight of the purified peptide was verified by mass spectrometry analysis. MS (MALDI TOF) found 2753.1 (M⁺) [theoretical 2752.041].

2. Preparation of Cy5Q-Asp-Glu-Val-Asp-Arg-Ser-Gly-Ser-Gly-Ser-Lys(Cy3)-Ala-Leu-Thr-OH (Compound B)

A dual-labelled peptide was synthesised as a control, using analogous methods.

2.1 Preparation of α-Fmoc-lysine(ε-Cy3)-OH (Compound 2)

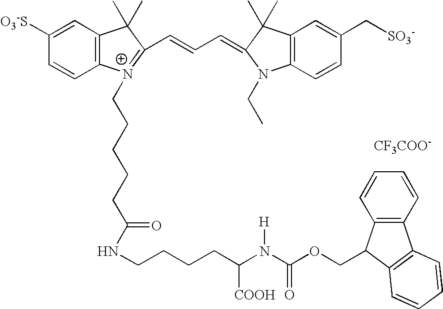

Cy3 mono free acid potassium salt (obtained from Amersham Pharmacia Biotech Ltd) (60 mg, 0.095 mmol) was dissolved in anhydrous dimethylsulphoxide (2 ml). To this was added O-(N-succinimidyl)-N,N,N′,N′-bis(tetramethylene)-uronium hexafluorophosphate (100 mg, 0.24 mmol) and N,N-diisopropylethylamine (80 μl). The reaction mixture was stirred at room temperature for 2 hours after which time negligible starting material remained by TLC (RPC₁₈, 1:1 methanol:water). The reaction mixture was slowly poured into diethyl ether to precipitate the product, Cy3 NHS ester, which was filtered off and dried in vacuo. The product was re-dissolved in anhydrous dimethylsulphoxide (2 ml) and N,N-diisopropylethylamine (80 μl) added. Fmoc-lysine-OH (50 mg, 0.14 mmol) was suspended in phosphate buffer (2 ml) and the suspension slowly added to the solution of Cy3 NHS ester. The reaction mixture was stirred at room temperature for 2 hours. TLC (RPC₁₈, 2:3 methanol:water) showed the disappearance of starting material and the formation of a new product spot. The product was purified by HPLC (Vydac protein peptide C₁₈ column (250×22.2 mm); flow rate 6 ml/min; gradient of 15 to 100% B over 60 mins (eluent A=0.1% TFA in water and eluent B=0.1% TFA in acetonitrile); dual wavelength detection at 254 and 550 nm), the retention time of the product was 27 mins. The fractions containing the desired product were pooled and the solvent removed under reduced pressure. The product was obtained as a magenta solid (70 mg). MS (MALDI TOF) found 979 (M⁺); [theoretical (C₅₂H₅₉N₄O₁₁S₂) 980].

2.2 Cy5Q-Asp-Glu-Val-Asp-Arg-Ser-Gly-Ser-Gly-Ser-Lys(Cy3)-Ala-Leu-Thr-OH (Compound B)

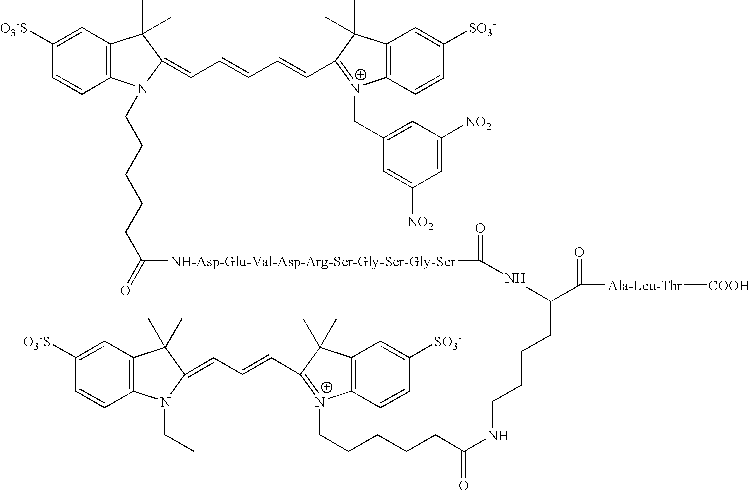

i) Ala-Leu-Thr-Wang Resin

Ala-Leu-Thr-Wang resin was synthesised using a commercially available Perkin-Elmer Model 431A automated peptide synthesiser and FastMoc™ chemistry, following the instrument manufacturer's recommended procedures throughout. The synthesis was performed on a 0.25 millimolar scale. The resin was removed from the peptide synthesizer and dried in vacuo.

ii) Fmoc-Lys(Cy3)-Ala-Leu-Thr-Wang Resin

α-Fmoc-lysine(ε-Cy3)-OH (Compound 2) (98 mg, 0.1 mmol) was coupled manually to Ala-Leu-Thr-Wang resin (250 mg, 0.1 mmol) using 7azabenzotriazol-1-yloxytris(pyrrolidino)phosphonium-hexafluorophosphate (PyAOP) (62.6 mg, 0.12 mmol) and diisopropylethylamine (122 μl, 0.7 mmol) in N-methyl pyrrolidone (NMP) at room temperature for 16 hrs. The reaction solvent was filtered off and the resin washed with NMP (3×10 ml). The resin was then capped using a standard capping solution (0.5M acetic anhydride, 0.125M diisopropylethylamine, 0.015M hydroxybenzotriazole) washed with N-methylpyrrolidone, dichloromethane and finally diethyl ether before drying in vacuo.

iii) Asp-Glu-Val-Asp-Arg-Ser-Gly-Ser-Gly-Ser-Lys(Cy3)-Ala-Leu-Thr-Wang Resin

Fmoc-Lys(Cy3)-Ala-Leu-Thr-Wang resin was returned to the Perkin-Elmer Model 431A automated peptide synthesiser and the final sequence of the peptide built using standard FastMoc™ chemistry, following the instrument manufacturer's recommended procedures throughout. The syntheses were performed on a 0.1 millimolar scale.

iv) Cy5Q-Asp-Glu-Val-Asp-Arg-Ser-Gly-Ser-Gly-Ser-Lys(Cy3)-Ala-Leu-Thr-OH (Compound B)

Cy5Q-NHS was coupled to Asp-Glu-Val-Asp-Arg-Ser-Gly-Ser-Gly-Ser-Lys(Cy3)-Ala-Leu-Thr-Wang resin in DMSO containing diisopropylethylamine at room temperature for 12 hrs. The purple resin was then filtered off, washed with DMSO, methanol and finally dichloromethane before drying in vacuo. The peptide was cleaved from the solid phase using a mixture of 95% trifluoroacetic acid:2.5% water:2.5% triisopropylsilane. The crude peptide obtained from the cleavage reaction was purified by conventional C-18 reverse phase HPLC using a linear gradient of water/acetonitrile (both containing 0.1% trifluoroacetic acid). After purification, the peptide was lyophilised to give a purple fluffy solid. The molecular weight of the purified peptide was verified by mass spectrometry analysis.

3. Assay for Protease Enzymes

3.1 Trypsin Cleavage Assay

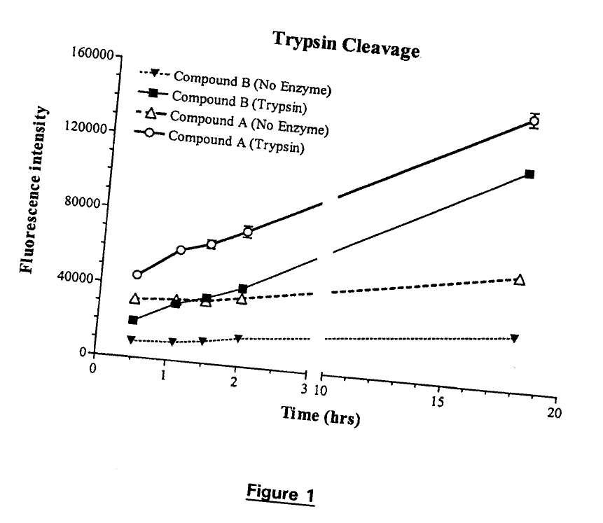

The protease substrate (Compound A: Cy5Q-Asp-Glu-Val-Asp-Arg-Ser-Gly-Ser-Gly-Ser-Cy3-Ala-Leu-Thr-OH) and a control dual labelled peptide (Compound B: Cy5Q-Asp-Glu-Val-Asp-Arg-Ser-Gly-Ser-Gly-Ser-Lys (Cy3)-Ala-Leu-Thr-OH) were diluted with PBS/0.005% Tween™ 20 to 2 μM. To 500 μl of each substrate, 1.5 units trypsin (20 μl volume) were added. The fluorescence intensity of 100 μl volumes (triplicate wells) in a black 96-well microplate (Dynex) was measured at intervals on a fluorescence plate reader using filter sets appropriate for Cy3 (535/10 nm for excitation and 569/10 nm for emission). Signals from ‘No enzyme’ blank samples (using 20 μl buffer in place of trypsin) were similarly recorded. The results are shown in FIG. 1.

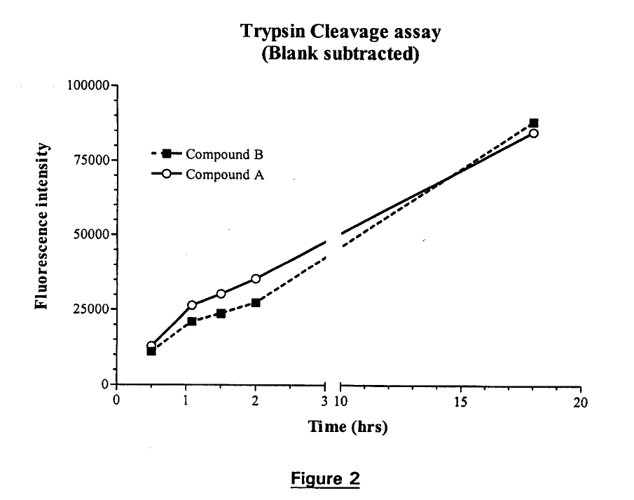

FIG. 2 shows the results after subtraction of the ‘no-enzyme’ blanks. Protease-catalysed hydrolysis of each substrate results in an increase in Cy3 signal, as the quenching effect of Cy5Q is reduced.

3.2 Endoproteinase AspN Cleavage Assay

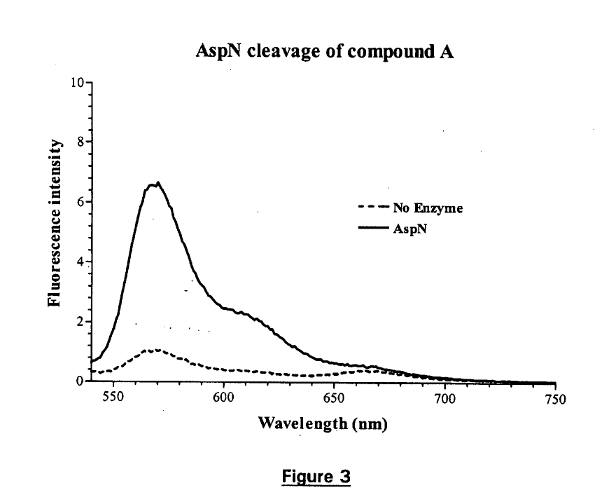

The fluorescence intensity of compound A (2 μM, 500 μl volume) in a quartz cuvette was recorded using a spectrofluorimeter. The sample was excited at 520 nm and emission over 540-750 nm recorded. AspN (100 ng, 50 μl volume) was added to the sample in the cuvette, which was stoppered, and kept dark at ambient temperature overnight. The fluorescence intensity was again recorded. The result is shown in FIG. 3. AspN-catalysed hydrolysis of Compound A shows an increase in Cy3 fluorescence, due to cleavage of the substrate, as the quenching effect of Cy5Q is reduced. Tween™ is a trademark of ICI Americas, Inc.

1. A compound comprising a peptide chain containing one or more dye molecules covalently bonded thereto, characterised in that at least one dye molecule is interposed in the amino acid sequence forming the peptide chain such that there is at least one amino acid covalently linked to and on each side of the said at least one dye molecule.

2. A compound according to claim 1 having the formula: 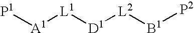
wherein D¹ is a dye molecule;
P¹ is an amino acid or a sequence comprising at least two amino acids and may include one or more functional groups for reaction with other groups;
P² is an amino acid or a sequence comprising at least two amino acids and may include one or more functional groups for reaction with other groups;
A¹ comprises an atom or a group suitable for attaching L¹ to P¹ by means of a covalent linkage;
B¹ comprises an atom or a group suitable for attaching L² to P² by means of a covalent linkage; and
L¹ and L² are each a linker chain and each independently contains from 1-20 linked atoms selected from the group consisting of carbon, nitrogen, oxygen, sulphur and phosphorus and combinations thereof and each L¹ and L² may be independently substituted by one or more groups selected from hydroxyl, halogen, C₁-C₄ alkyl, C₁-C₄ alkoxy, aryl, heteroaryl and aralkyl.

3. A compound according to claim 1 having the formula: 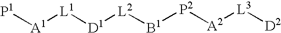
wherein D¹ and D² may be the same or different and are each a dye molecule;
P¹, P², A¹ and B¹ are as hereinbefore defined;
A² comprises an atom or a group suitable for attaching L³ to P² by means of a covalent linkage; and
L¹, L² and L³ are each as hereinbefore defined for L¹ and L².

4. A compound according to claim 1 having the formula: 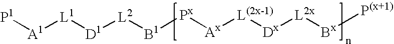
wherein n is an integer and x is (n+1); and
P¹ to P^{(x+1)}, A¹ to A^{x}, B¹ to B^{x}, L¹ to L^{2x} and D¹ to D^{x} are as hereinbefore defined for P¹, P², A¹, B¹, L¹, L², L³, D¹ and D².

5. A compound according to claim 4 wherein the dye-labelled peptide chain containing more than one interposed dye may also include a dye attached to a terminal amino acid.

6. A compound according to claim 4 having the formula: 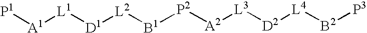
wherein P¹, P², P³, A¹, A², B¹, B², L¹, L², L³, L⁴, D¹ and D² are as hereinbefore defined for P¹, P², A¹, B¹, L¹, L², L³, D¹ and D².

7. A compound according to claims 2 to 6 wherein L¹ to L^{2x} may be selected from linear or branched C₁₋₂₀ alkyl chains, which may optionally contain one or more ether linkages, one or more amide linkages, one or more unsaturated groups, including —CR^{a}═CR^{a}—, —C≡C—, and phenylene which may be substituted with 1, 2, 3 or 4 substituents independently selected from hydroxyl, halogen, C₁-C₄ alkyl, C₁-C₄ alkoxy, aryl, heteroaryl and aralkyl and R^{a} is selected from hydrogen and C₁-C₄ alkyl, and wherein x is hereinbefore defined.

8. A compound according to claims 3 to 7 wherein the distance R between the centres of D¹ and D² (or D^{x} and D^{(x±1)}, where D¹ and D² (or D^{x} and D^{(x±1)}) are respectively donor and acceptor dyes in an energy transfer relationship and wherein x is hereinbefore defined, may be from 10 to 80 Angstroms.

9. A compound according to claim 8 wherein the relative orientation of the transition moments of D^{x} and D^{(x±1)} during the excited state lifetime of the donor and the proximity of the donor and the acceptor dyes are such that there is sufficient energy transfer.

10. A compound according to claims 2 to 9 wherein D¹ (and/or D² to D^{x} if present), is a fluorescent dye, wherein x is hereinbefore defined.

11. A compound according to claims 3 to 9 wherein one or more of D¹ to D^{x} is a fluorescent dye and the remaining D¹ to D^{x} is a non-fluorescent or quenching dye, wherein x is hereinbefore defined.

12. A compound according to claim 11 wherein said fluorescent dye and said non-fluorescent dye are at adjacent dye positions.

13. A compound according to any of claims 10 to 12 wherein said fluorescent dye is selected from the group consisting of fluoresceins, rhodamines, coumarins, bis-pyrromethine boron difluoride dyes and cyanine dyes.

14. A compound according to claim 11 or claim 12 wherein said non-fluorescent dye is selected from the group consisting of 2,4-dinitrophenyl (DNP), 4-(4-dimethylaminophenyl)azobenzoic acid (DABCYL) and cyanine dyes.

15. A method for detecting the presence of a biological material which method comprises use of a compound according to any one of claims 1 to 14.

16. An assay method which comprises:
a) separating two components which are in an energy transfer relationship each of said components comprising a peptide chain, the first component being labelled with a fluorescent donor dye and the second component being labelled with a non-fluorescent acceptor dye wherein at least one of said dye molecules is interposed in the amino acid sequence forming the peptide chain such that there is at least one amino acid covalently linked to and on each side of the said at least one dye molecule; and
b) detecting the presence of the first component by measuring emitted fluorescence.

17. A method according to claim 16 wherein said assay method is a proteolytic enzyme cleavage assay.

18. Use of a compound according to any one of claims 1-14 for analysis or detection.
